(19) United States
(12) Patent Application Publication
(10) Pub. No.: US 2013/0210657 A1
(43) Pub. Date: Aug. 15, 2013

(54) Method of Determining Risk of Autism Spectrum Disorder

(71) Applicant: THE HOSPITAL FOR SICK CHILDREN, (US)

(72) Inventor: Stephen Scherer, Toronto (CA)

(73) Assignee: THE HOSPITAL FOR SICK CHILDREN, Toronto (CA)

(21) Appl. No.: 13/750,541

(22) Filed: Jan. 25, 2013

Related U.S. Application Data
(60) Provisional application No. 61/590,591, filed on Jan. 25, 2012.

(51) Int. Cl. C12Q 1/68

(52) U.S. Cl. CPC C12Q 1/6883
USPC 506/9; 435/6.11; 436/86

(57) ABSTRACT

A method of assessing risk in a human subject of ASD is provided comprising the step of identifying in a nucleic acid-containing sample obtained from the human subject copy number variations associated with SHANK1. Determination of copy number variations associated with SHANK1 is indicative of a risk of ASD in the human subject.

This application claims the benefit of U.S. Provisional Patent Application No. 61/590,591, filed on Jan. 25, 2012, and incorporates such provisional patent application in its entirety by reference.

### FIELD OF THE INVENTION

The present invention relates to a method of assessing in a human subject, the risk of having Autism Spectrum Disorder (ASD) using novel biomarkers.

### BACKGROUND OF THE INVENTION

Autism is the prototypic form of a group of conditions, the ‘autism spectrum disorders’ (ASD), which share common characteristics (impairments in socialization, communication and repetitive interests and behaviors), but differ in developmental course, symptom pattern, cognitive and language abilities. Other ASD subtypes include Asperger disorder (less severe language and cognitive deficits) and Pervasive Developmental Disorder Not Otherwise Specified (PDD-NOS; sub-threshold symptoms and/or later onset). Sub clinical forms of ASD are often characterized as Broader Autism Phenotype (BAP). Twin and family studies provide evidence for the importance of complex genetic factors in the development of both sporadic and inherited forms of idiopathic autism. An enigma in ASD is the 4:1 male to female gender bias, which may rise to 11:1 when considering Asperger disorder.

Rare copy number variations (CNVs) and sequence-level mutations have been identified as etiologic factors in ASD. De novo CNVs are observed in 5-10% of ASD cases. A relative enrichment of CNVs disrupting synaptic complex genes is observed, with NLGN3, NLGN4, NRXN1, NRXN3, SHANK2 and SHANK3 being identified as highly-penetrant susceptibility loci for ASD and intellectual disability (ID).

In order to further understand the etiology of neurodevelopmental disorders such as ASD, it would be desirable to identify additional biomarkers of such disorders.

### SUMMARY OF THE INVENTION

It has now been determined that copy number variations or other sequence variations associated with the SHANK1 gene are indicative of risk of ASD.

Accordingly, in one aspect of the invention, a method of assessing risk of ASD in a human subject is provided. The method comprises identifying in a nucleic acid-containing sample obtained from the human subject copy number variations associated with SHANK1. A determination of copy number variations associated with SHANK1 is indicative of a risk of ASD in the human subject.

This and other aspects of the invention are described in the detailed description by reference to the following figures.

### BRIEF DESCRIPTION OF THE FIGURES

FIG. 1 illustrates the SHANK1 nucleotide (A) and protein (isoform 1) (B) sequences.

FIG. 2 illustrates rare deletions at SHANK1 locus in the two ASD families. Chromosomal position of rare deletions of SHANK1 and adjacent genes in ASD. The accurate coordinates for Family 1 were mapped by sequencing across the breakpoints: Chr 19: 55,872,189-55,935,995 (hg18). The de novo deletion of Family 2 was detected by microarray with coordinates of Chr 19: 55,808,307-55,871,709 (hg18).

FIG. 3. Location of rare missense variations and deletions in SHANK1 identified in ASD patients. Domain structure of the SHANK1 protein is shown. Domain name abbreviations: ANK: ankyrin repeats domain; SH3: Src homology 3 domain; PDZ: postsynaptic density 95/Discs large/zona occludens-1 homology domain; SAM: sterile alpha motif domain.

FIG. 4. Pedigrees of ASD families with rare non-synonymous variants. Circles and squares denote females and males, respectively, whereas arrows highlight the index proband in each family. Black filled objects indicate ASD diagnosis, unfilled symbols signify unaffected family members. N/A denotes individuals from whom no DNA was available for testing.

FIG. 5. Karyotypes and FISH testing results of chromosome 5 and 19 from family 1. Figure displays karyotpes and results from FISH testing of chromosomes 5 and 19 in three individuals from family 1: female deletion carrier II-4 (A), male ASD proband III-5 with deletion (B) and female individual III-3 without deletion (C). The SpectrumOrange probe hybridized with one signal to each of two chromosomes 5 at band 5q31.3 as expected in II-4, III-5 and III-3. SpectrumGreen probe hybridized with one signal to one chromosome 19 at band 19q13.33 and the 64 kb deletion was confirmed in II-4 and III-5. There was no dim and consistent orange doublet probe hybridized with one signal to each of two chromosomes 19 at band 19q13.33 as expected. Chromosomes 5 and 19 were confirmed by G-to-FISH. Karyotypes are following;

(A) 46,XX.ish del(19)(q13.33q13.33)(G248P87495E12-)

(B) 46,XY.ish del(19)(q13.33q13.33)(G248P87495E12-)

(C) 46,XX.ish 5q31.3(G248P80200H8x2),19q13.33(G248P87495E12x2).

FIG. 6. Results of Linkage analysis of Family 1. Plot of LOD score results from parametric linkage analysis of Family 1, conducted using MMLS (maximized maximum LOD score) approach. Highest observed LOD score was with the dominant model using 95% penetrance and 0.01 disease allele frequency. The maximum LOD score was 1.726. This maximum LOD score was equal to the score which could be obtained with this pedigree, under ideal conditions, based on 1000 simulations (performed using SLINK²⁴). The score was reached on 5 different chromosomes: 5, 10, 15, 17 and 19. No signal was seen on the X chromosome.

FIG. 7 illustrates the pedigree of multi-generation Family 1 carrying a rare CNV that deletes one copy of the SHANK1 gene. Individuals with ASD and BAP (Broader Autism Phenotype) are indicated by filled symbols and striped symbol, respectively. The proband is indicated by an arrow. Wt indicates individuals having the typical copy number of two at the SHANK1 locus and NA indicates unavailability of DNA.

FIG. 8 illustrates the pedigree of Family 2 in which an ASD proband II-1 has a heterozygous deletion of SHANK1 and SYT3.

### DETAILED DESCRIPTION OF THE INVENTION

A method of assessing risk of Autism Spectrum Disorder (ASD) in a human subject is provided. The method comprises identifying in a nucleic acid-containing sample obtained from the human subject copy number variations associated with SHANK1. A determination of copy number variations associated with SHANK1 is indicative of a risk of ASD in the human subject.

The term “ASD” or “Autism Spectrum Disorder” is used herein to refer to autism, Asperger syndrome, Childhood disintegrative disorder, Rett syndrome, Pervasive Developmental Disorder Not Otherwise Specified (PDD-NOS) and Broader Autism Phenotype (BAP).

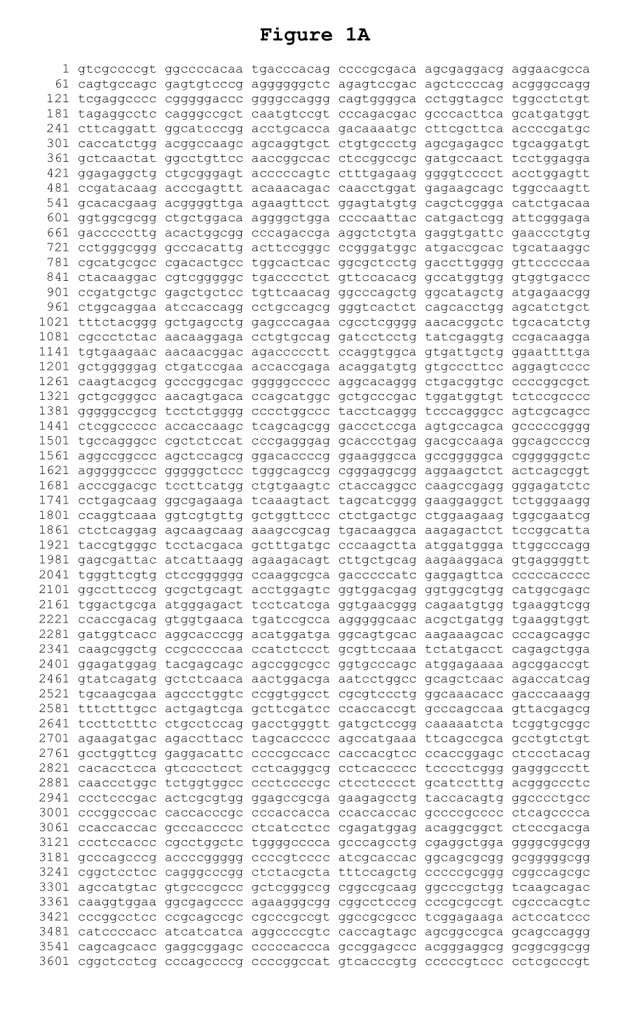

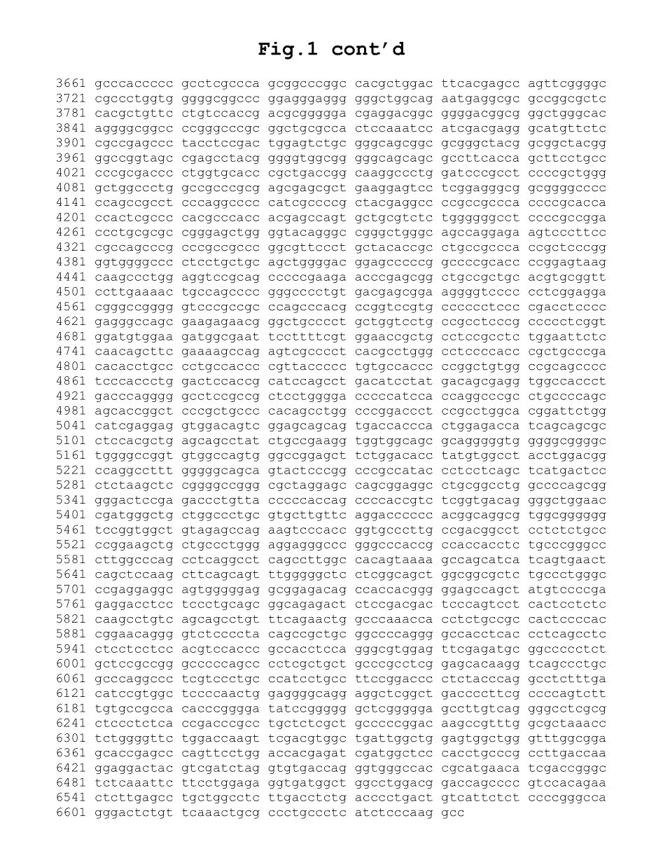

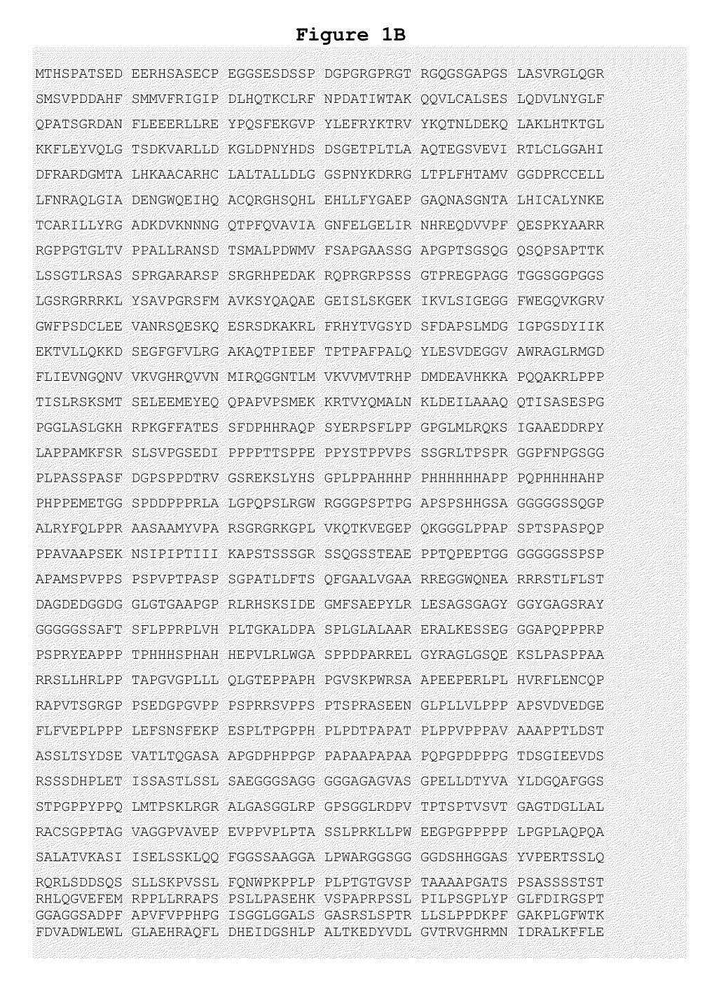

The term “SHANK1” refers to the gene that encodes a protein known as SH3 and multiple ankyrin repeat domains protein 1 (shank1), including natural variants and isoforms, e.g. isoforms 1, 2 and 3, thereof. This term encompasses the human gene sequence as set out in FIG. 1A, and functionally equivalent variants thereof. The term “functionally equivalent variant” refers to a gene sequence that may vary from the identified sequence due to degeneracy in the nucleic acid sequence, or codon insertions, deletions or substitutions, but which encodes a functional protein product. The amino acid sequence of isoform 1 of shank 1 is provided in FIG. 1B. Isoform 1 differs by deletion of amino acids 1-613, while isoform 3 differs by deletion of amino acids 646-654.

In the present method of determining risk in a human subject of ASD, a biological sample obtained from the subject is utilized. A suitable biological sample may include, for example, a nucleic acid-containing sample or a protein-containing sample. Examples of suitable biological samples include saliva, urine, semen, other bodily fluids or secretions, epithelial cells, cheek cells, hair and the like. Although such non-invasively obtained biological samples are preferred for use in the present method, one of skill in the art will appreciate that invasively-obtained biological samples, may also be used in the method, including for example, blood, serum, bone marrow, cerebrospinal fluid (CSF) and tissue biopsies such as tissue from the cerebellum, spinal cord, prostate, stomach, uterus, small intestine and mammary gland samples. Techniques for the invasive process of obtaining such samples are known to those of skill in the art. The present method may also be utilized in prenatal testing for the risk of ASD using an appropriate biological sample such as amniotic fluid and chorionic villus.

In one aspect, the biological sample is screened for SHANK1 in order to detect mutations in the genome associated with ASD. It may be necessary, or preferable, to extract nucleic acid from the biological sample prior to screening the sample. Methods of nucleic acid extraction are well-known to those of skill in the art and include chemical extraction techniques utilizing phenol-chloroform (Sambrook et al., 1989), guanidine-containing solutions, or CTAB-containing buffers. As well, as a matter of convenience, commercial DNA extraction kits are also widely available from laboratory reagent supply companies, including for example, the QIAamp DNA Blood Minikit available from QIAGEN (Chatsworth, Calif.), or the Extract-N-Amp blood kit available from Sigma (St. Louis, Mo.).

Once an appropriate nucleic acid sample is obtained, it is subjected to well-established methods of screening, such as those described in the specific examples that follow, to detect genetic mutations in SHANK1 which are indicative of ASD. These mutations include genomic copy number variations (CNVs), such as gains and deletions of segments of DNA, for example, gains and deletions of segments of DNA greater than about 10 kb, such as DNA segments greater than 50 kb. Gene mutations or CNVs “associated with” ASD include CNVs in both coding and regulatory regions of the SHANK1 gene. In a preferred embodiment, gene mutations in the form of CNVs which reduce or inhibit SHANK1 expression are indicative of a risk of ASD. The CNVs may be inherited or de novo.

To determine risk of, or to diagnose ASD, in a human subject, it may be advantageous to screen for multiple CNVs that are associated with ASD applying array technology. In this regard, genomic sequencing and profiling, using well-established techniques as exemplified herein in the specific examples, may be conducted for a subject to be assessed with respect to ASD risk/diagnosis using a suitable biological sample obtained from the subject. Identification of one or more CNVs associated with ASD would be indicative of a risk of ASD, or may be indicative of a diagnosis of ASD. This analysis may be conducted in combination with an evaluation of other characteristics of the subject indicative of ASD, including for example, phenotypic characteristics.

In another aspect, a method for determining risk of ASD in a subject is also provided in which the expression or activity of the SHANK1 protein product, e.g. a SH3 and multiple ankyrin repeat domains protein 1, including isoforms thereof, is determined in a biological protein-containing sample obtained from the subject. Abnormal levels of the gene product or abnormal levels of the activity thereof, i.e. reduced or elevated levels, in comparison with levels that exist in healthy non-ASD subjects, are indicative of a risk of ASD, or may be indicative of ASD. As one of skill in the art will appreciate, standard assays may be used to identify and quantify the presence and/or activity of a selected gene product.

In a further aspect, identification of missense mutations in SHANK1 may also be indicative of risk of ASD. In this regard, a missense mutation may result in a reduction of SHANK1 expression, or in the expression of a protein product with altered activity, e.g. reduced activity, or a non-functional protein. Accordingly, in addition to detection of the mutation in a nucleic acid sample of the patient, risk of ASD may also be assessed by detection of an altered, e.g. reduced level and/or activity of the gene product of SHANK1.

Embodiments of the invention are described by reference to the following specific example which is not to be construed as limiting.

### Example 1

### Materials and Methods

The ASD patient dataset comprised 1,158 unrelated Canadian individuals (898 males and 260 females) and 456 unrelated cases (362 males and 94 females) from Europe. The patients had clinically well characterized ASD diagnosed by expert clinicians based on the Autism Diagnostic Interview—Revised (ADI-R) and/or the Autism Diagnostic Observation Schedule (ADOS) as set out in Risi et al. J Am Acad Child Adolesc Psychiatry 2006; 45:1094-103, the relevant contents of which are incorporated herein by reference. Canadian cases were recruited from five different sites: The Hospital for Sick Children, Toronto, Ontario; McMaster University, Hamilton, Ontario; Memorial University of Newfoundland, St. John's, Newfoundland; University of Alberta, Edmonton, Alberta and the Montreal Children's Hospital of the McGill University Health Centre, Montreal, Quebec, Canada. The European ASD cases were recruited by the PARIS (Paris Autism Research International Sibpair) study and several other sites at specialized clinical centers dispersed in France, Sweden, Germany, Finland and UK. In Sweden, for some cases, the Diagnostic Interview for Social and Communication Disorders (DISCO-10) was applied instead of the ADI-R. The ID patient dataset consisted of 185 mostly French Canadians (98 males and 87 females) and 155 German non-syndromic ID cases (93 males and 62 females). Further descriptions of these datasets and the assessment procedures used are described in Hamdan et al. (Biol Psychiatry 2011; 69:898-901) and Berkel et al. (Nat Genet 2010; 42:489-91).

Institutional ethical review board approval was obtained for the study, and informed written consent was obtained from all participants.

### CNV Detection and Validation

To assess the presence of CNVs on a genome-wide scale, DNA from the Canadian ASD dataset was genotyped at The Centre for Applied Genomics, Toronto with one of three high-resolution microarray platforms: Affymetrix GeneChip SNP 6.0, Illumina Infinium 1M single SNP or Agilent SurePrint G3 Human CGH 1x1M. CNVs were analyzed using published methods (Pinto et al. Nat Biotechnol 2011. 29:512-20, the relevant contents of which are incorporated herein by reference). Independent validation of the deletion at the SHANK1 locus in Family 1 was performed with SYBR Green based real-time quantitative PCR (qPCR), with two independent primer pairs at the SHANK1 locus, and at the FOXP2 locus as a negative (diploid) control.

DNA from the European ASD case dataset was genotyped at the Centre National de Genotypage (CNG), at the Institut Pasteur using the Illumina Human 1M-Duo BeadChip. CNVs were analyzed as above. Validation of the array CNV calls was performed with qPCR in a similar way as described above, with two independent primer pairs at the SHANK1 locus, and at exon 18 locus of SHANK1 as a negative (diploid) control. All primers are listed in the Table that follows:

TABLE 1
List of primers used for sequencing of SHANK1

| Primer for sequencing | Primer sequence |
|---|---|
| SHANK1-EXON1F | CAGCCTCCTTCCTGCCTATC |
| SHANK1-EXON1R | GGAGGATACCCAGCACCAGT |
| SHANK1-EXON2F | GTCCACTGGTGCTGGGTATC |
| SHANK1-EXON2R | GCAGAACAGATGGTAATTTGAACTC |
| SHANK1-EXON3F | ATCTACCGCCTAGACCAAGGTT |
| SHANK1-EXON3R | TGTGGTACAGCATCCCAAGTTA |
| SHANK1-EXON4F | TTTCAATGGCGTATGTGACTCC |
| SHANK1-EXON4R | CCCTTGGACAGCAATGTGTTT |
| SHANK1-EXON5F | TCTGCATTCACATCCATTCC |
| SHANK1-EXON5R | CTGACAAGGGTGACAATAGGG |
| SHANK1-EXON6F | AGTCCCACATTGTTCACACG |
| SHANK1-EXON6R | CTTAGGGTCTTTCTGCCTTCAC |
| SHANK1-EXON7F | CTTGGAATGACTGAACATTTGG |
| SHANK1-EXON7R | GATGGATGGATGGAGGAATG |
| SHANK1-EXON8F | GCTGCTGTCCTCAGTGGTG |
| SHANK1-EXON8R | CCCTCTGTCTTCTTCCAGCTC |
| SHANK1-EXON9F | TTGTCGGAGTGGAAGGTTTG |
| SHANK1-EXON9R | GGCATGAGGGAGAAAGACAG |
| SHANK1-EXON10F | TCTCTCCCACCATCTCTTGC |
| SHANK1-EXON10R | TTGGATGAGGGCCTACAGAG |
| SHANK1-EXON11F | CTGATGCACCGTCCTCTTC |
| SHANK1-EXON11R | ATGGTCCTCCAAGCCTCAAG |
| SHANK1-EXON12F | GCTGGTAACTGTGGGAATGC |
| SHANK1-EXON12R | TTTCTGCAGGGTGACAACAG |
| SHANK1-EXON13F | CCTAGGATTCCCACGTCCAC |
| SHANK1-EXON13R | AAGCTAATTCTGGCTTATCC |
| SHANK1-EXON14F | CTGTGCAGTCATGTGCAGTG |
| SHANK1-EXON14R | AAACCTCAGCTCTGGTCGTG |
| SHANK1-EXON15F | CTGAATGGATGGGTGGATG |
| SHANK1-EXON15R | GGGCTCAGACCCAAGTCAC |
| SHANK1-EXON16F | GTGAGGCCTCCGTGACTTG |
| SHANK1-EXON16R | AACTGGGCAGCCAGATCC |
| SHANK1-EXON17F | GGAGGGAGAGGAACATAGCC |
| SHANK1-EXON17R | CACGGAGAAGCAGTGCTAGG |
| SHANK1-EXON18F | TTCCCTAGCACTGCTTCTCC |
| SHANK1-EXON18R | CCCTTCCCAGAGACACACAC |
| SHANK1-EXON19F | GAGTGGTGAGTGGGCACAG |
| SHANK1-EXON19R | ACAATCTCCCAGCCCAGTG |
| SHANK1-EXON20F | GGGAGATTGTGTCTCCAAGC |
| SHANK1-EXON20R | GAAACCCTAGGATGTGTGTCG |
| SHANK1-EXON21F | CTTCCACCGTCTTCACACTG |
| SHANK1-EXON21R | GGATTCATGGCCAAGTTCAC |
| SHANK1-EXON22_1F | TGCAGTGCACAACCTGTACC |
| SHANK1-EXON22_1R | GGCAGCTGGAAATAGCGTAG |
| SHANK1-EXON22_2F | CTCCCGAGATGGAGACAGG |
| SHANK1-EXON22_2R | GACTCCAGTCGGAGGTAGGG |
| SHANK1-EXON22_3F | CTGTTCCTGTCCACCGACG |
| SHANK1-EXON22_3R | GCTTTTCGAAGCTGTTGGAG |
| SHANK1-EXON22_4F | AGGGCCAGCGAAGAGAAC |
| SHANK1-EXON22_4R | CCGGAGCTTAGAGGGAGTC |
| SHANK1-EXON22_5F | AGCCTATCTGCCGAAGGTG |
| SHANK1-EXON22_5R | CCAACCTGGTTTCTGTTTCC |
| SHANK1-EXON23F | CCCTACCCTTATGTCTCTCCTC |
| SHANK1-EXON23R | CCCTCTGTAATTTCTCCTATCC |

### Control CNV Datasets

The SHANK1 locus was also examined for CNVs in published data from 2,026 healthy individuals from the Children's Hospital of Philadelphia and from 2,493 controls genotyped at the University of Washington⁸ and in microarray data analyzed by our group from 10,603 population based controls.^{4,5,9,10} This latter dataset included 1,123 controls from northern Germany,¹¹ 1,234 Canadian controls,¹² 1,120 population controls from Ontario,¹³ 1,056 HapMap samples,¹⁴ 4,783 controls from the Wellcome Trust Case Control Consortium (WTCCC)¹⁵ and 1,287 controls recruited by the Study of Addiction: Genetics and Environment (SAGE) consortium.¹⁶ Control samples were predominantly of European ancestry appropriate for comparison with the ASD case datasets. The Database of Genomic Variants (DGV; http://projects.tcag.ca/variation) was also examined for previously reported CNVs at the SHANK1 locus in the general population.

### Sequencing and Mutation Screening Methods

509 of the 1,158 ASD individuals and 340 individuals with ID were screened for mutations using Sanger-based sequencing. All coding exons and intron-exon splice sites of SHANK1 were sequenced. Primer3 software v. 0.4.0 (http://frodo.wi.mit.edu/primer3) was used to design PCR primers. PCRs were performed using standard conditions, and products were purified and sequenced directly using the BigDye Terminator sequencing (Applied Biosystems, Foster City, Calif., USA). Variant detection was performed using SeqScape software from Applied Biosystems. Novel variants detected in the cases, not previously reported in the Single Nucleotide Polymorphism Database (dbSNP) build 130, were validated by re-sequencing the proband and samples from both parents and from siblings, when available. All primers are listed in the following Table.

TABLE 2
q-PCR primers for CNV validation/breakpoint
mapping

| Primer for qPCR | Primer sequence |
|---|---|
| 1F | GTAACAGGGAGAATCAGCCAAG |
| 1R | AAAGATGGAGAAGGGAGACACA |
| 2F | TTCTTTCAGATTTCGGCTCCA |
| 2R | GAGACAGACAGTAAACAAGCAAGCA |
| 3F | TACTCTGCTTGGCTTTCTGTCC |
| 3R | TTCCACTTGCCACTTCTCTACTG |
| 4F | TTGCACTGATGGTCTGTTGAG |
| 4R | GGGTCAAAGCAAACTTCATTTC |
| 5F | GAAAGCATCTGAGGGAGAGAAG |
| 5R | TCTTCACATGAGGGTCAGGAT |
| 6F | GAGTCAGCCTTCCATCAGAAAT |
| 6R | TCTGACCTCTGGTTGGCTATAAG |
| 7F | CGTATTCATTCACGCACCAG |
| 7R | ACGTGACAATGATGCTGTTAGG |
| 8F | ACCCAAGCATGAAGTGAAATAGC |
| 8R | TCTTTACGTGGGTGAATTGCAT |
| 9F | TTCAGCAATTCCCACCCAGT |
| 9R | GGGTATGCAGTGAAAGAGCAGAA |
| 10F | TCAACAGACCATCAGTGCAAG |
| 10R | GCCTACCTCAGTGGCAAAGA |
| 11F | GACTGCCGCTCCAAAGTC |
| 11R | GAAGGACGCTCGTAACTTGG |
| 12F | GGGAAGGGCCTATTCTGG |
| 12R | ACAGTCCCCATCCAATCG |

### TaqMan Assay

For the rare SHANK1 sequence missense variants identified in ASD and ID, TaqMan assays were performed to estimate their frequency in 285 control individuals of European ancestry from the Ontario Population Genomics Project control collection (138 males and 147 females) using the Applied Biosystems 7900HT real-time PCR system.

### Exome Sequencing

For two of the ASD patients in family 1 (III-5 and IV-3), paired-end exome sequencing was performed using Life Technologies SOLiD5500 (Life Technologies, Foster City, Calif., USA) sequencing platform. Target enrichment was performed utilizing the Agilent SureSelect 50 Mb human all exon capture kit (Agilent Technologies, Santa Clara, Calif., USA). Protocols for sequencing and target capture followed specifications from the manufacturers. BFAST (Homer et al. PLoS One 2009; 4:e7767) was used to map the generated paired end reads to the reference human genome (UCSC's hgl9). Duplicate pair end reads were removed using MarkDuplicates (Picard tools version 1.35; http://picard.sourceforge.net) and the subsequent duplicated-free alignments were refined using local realignment in colourspace implemented in SRMA version 0.1.15 (Homer et al. Genome Biol 2010; 11:R99). Calling of indels and SNPs was performed using GATK version1.0.5506 and recommended parameters (DePristo et al. Nat Genet 2011; 43:491-8). SIFT 4.0.3 (Ng et al. Nucleic Acids Res 2003; 31:3812-4) was used to annotate the variant calls to determine if amino acid substitutions were predicted to be deleterious.

The nonsense variant in PCDHGA11 was validated by Sanger sequencing. All primers are listed in the table below.

TABLE 3
Primers for PCDHGA11 sequencing

| Primer for sequencing | Primer sequence |
|---|---|
| PCDHGA11-EXON1.1F | AACCAACCAGCTCGAGAAAC |
| PCDHGA11-EXON1.1R | CACGCGATATACGGACTGTG |
| PCDHGA11-EXON1.2F | AGAAAGAGGCTGCTCACCTG |
| PCDHGA11-EXON1.2R | TCTGGCCTGAATCTTTGTCC |
| PCDHGA11-EXON1.3F | ATGCCCTACAATCCTTCGAC |
| PCDHGA11-EXON1.3R | AAATTGAGAGCCTCATACACTG |
| PCDHGA11-EXON2F | TCAGCTTGCTCACTGTGGTC |
| PCDHGA11-EXON2R | CCTGAACAGTCAGGGCAGTC |
| PCDHGA11-EXON3F | AAGTGCCTCCTACCTTGCTG |
| PCDHGA11-EXON3R | TTGGAATTGTGGGTCCTTTC |
| PCDHGA11-EXON4F | TTGTGAAGAGAGACTACCTTGGTG |
| PCDHGA11-EXON4R | TGGGTGCAGGTAAGGAGAAG |

TABLE 4
Primer to validate the stop mutation in PCDHGA11

| | |
|---|---|
| PCDHGA11-1F | TGCTGATGGTTAATGCAACG |
| PCDHGA11-2R | CTCTGGACCAACTCCCTGTC |

### Fluorescence In Situ Hybridisation

Chromosome metaphases were prepared according to standard protocols from primary blood samples. Metaphase FISH were performed using two fosmid probes (hg18 co-ordinates): G248P80200H8 (chr5:140,769,097-140,810,244, SpectrumOrange) overlapping the Y313X nonsense mutation in PCDHGA11 and G248P87495E12 (chr19: 55,874,318-55,921,609, SpectrumGreen) residing within the 63.8 kb deletion disrupting SHANK1.

### Linkage Analysis

Eleven individuals from Family 1 (I-1, I-2, II-2, II-4, II-5, III-1, III-2, III-3, III-5, III-6 and IV-3) were genotyped using the Illumina Omni 2.5M-quad BeadChip microarray platform. Genotype information from 5,629 SNPs was used for linkage analysis. These markers were selected to have high call rate, high MAF, no Mendelian errors, low pairwise LD between them, and genotype proportions consistent with Hardy Weinberg equilibrium. Markers with ambiguous alleles were also removed. Additionally, only markers which were present in the HapMap3 release 28 CEU population were included, so that allele frequencies could be properly estimated. Parametric linkage analysis with the MMLS (maximized maximum LOD score) method was performed using the program Merlin (Abecasis et al. Nat Genet 2002; 30:97-101). This analysis is appropriate when the correct method of inheritance is not known and is more powerful than non-parametric analysis, since it uses all individuals in the pedigree and not just the affected ones. In the MMLS method, the pedigree is analyzed for linkage under several different inheritance models (dominant and recessive with varying penetrance) and the model with the maximum LOD score was chosen.

### Results—Deletions at the SHANK1 Locus

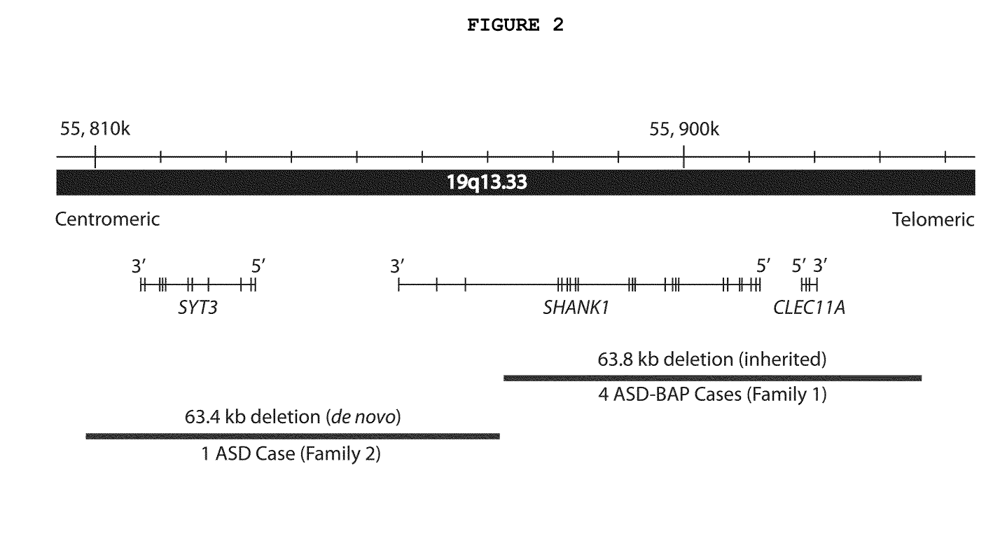

Initially, 1,158 Canadian individuals with ASD were examined using high-resolution microarray scanning and a hemizygous microdeletion at chromosome 19q13.33 in ASD proband III-5 in Family 1 was identified. The deletion was determined to be 63.8 kilobases (kb) eliminating exon 1 to 20 of SHANK1, and the neighboring CLEC11A gene coding for a growth factor for primitive hematopoietic progenitor cells (FIG. 2). Subsequent genotyping in Family 1 revealed that the deletion was also present in males I-1, IV-1 and IV-3, as well as females II-4 and III-2.

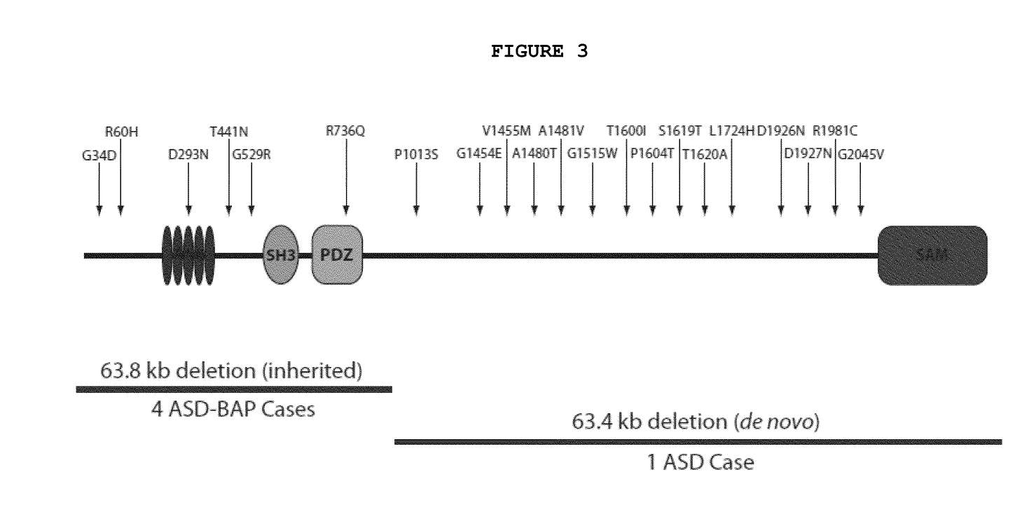

In separate experiments, 456 individuals from Europe with ASD were examined using microarrays and a 63.4 kb hemizygous CNV was identified in individual F2-II-1 from Family 2 deleting the last three exons of SHANK1 and the entire centromeric synaptotagmin-3 (SYT3) gene, with a role in Ca(2+)-dependent exocytosis of secretory vesicles (FIG. 2). Haplotype analysis revealed the deletion resided on the chromosome originating from the mother (who was shown to carry two copies of SHANK1). The deletion was not in F2-II-3. No equivalent deletion to those described in Family 1 or 2, was observed in 15,122 control individuals or in the Database of Genomic Variants (FIG. 3). Taken together, the frequency of deletions at the SHANK1 locus is significantly higher in ASD cases compared to controls (2/1,614 cases vs. 0/15,122 controls; Fisher's Exact test two-tailed p=0.009). No other obvious potentially etiologic CNV was observed in any of the individuals with ASD in Family 1 or 2 as set out in the Tables below. Therefore, at this resolution of analysis the rare deletion of common segments of SHANK1 were the only common events observed between the two unrelated ASD families.

TABLE 5
CNVs detected in III-5 with Agilent SurePrint G3 Human CGH 1x1M microarray

| Number | Cytoband | Start (Build 36) | Stop (Build 36) | Size (bp) | Type | Class^{a} |
|---|---|---|---|---|---|---|
| 1 | 19q13.33 | 55,872,843 | 55,934,778 | 61,936 | Loss | likely pathogenic |
| 2 | 4p15.33 | 10,984,237 | 10,989,599 | 5,363 | Loss | likely benign |
| 3 | 1p31.1 | 72,538,943 | 72,557,598 | 18,656 | Gain | Normal |
| 4 | 1q21.1 | 147,306,104 | 147,645,031 | 338,928 | Gain | Normal |
| 5 | 1q21.3 | 150,822,873 | 150,851,639 | 28,767 | Loss | Normal |
| 6 | 1q24.2 | 167,493,568 | 167,508,098 | 14,531 | Gain | Normal |
| 7 | 2p22.3 | 34,551,022 | 34,590,197 | 39,176 | Loss | Normal |
| 8 | 2p13.2 | 73,706,527 | 73,764,697 | 58,171 | Gain | Normal |
| 9 | 2p11.1, 2p11.2 | 88,913,881 | 91,158,469 | 2,244,589 | Gain | Normal |
| 10 | 2q13 | 110,200,015 | 110,341,133 | 141,119 | Gain | Normal |
| 11 | 2q37.3 | 242,571,023 | 242,597,073 | 26,051 | Loss | Normal |
| 12 | 3q26.1 | 164,036,448 | 164,108,151 | 71,704 | Gain | Normal |
| 13 | 3q29 | 194,354,305 | 194,367,150 | 12,846 | Gain | Normal |
| 14 | 3q29 | 196,835,213 | 196,961,438 | 126,226 | Gain | Normal |
| 15 | 4p15.1 | 34,457,448 | 34,506,497 | 49,050 | Loss | Normal |
| 16 | 4q13.2 | 69,069,451 | 69,166,014 | 96,564 | Loss | Normal |
| 17 | 5p15.33 | 775,994 | 830,154 | 54,161 | Loss | Normal |
| 18 | 5q31.3 | 140,203,240 | 140,216,724 | 13,485 | Loss | Normal |
| 19 | 5q33.2 | 155,410,853 | 155,421,643 | 10,791 | Loss | Normal |
| 20 | 5q35.3 | 180,342,660 | 180,359,177 | 16,518 | Gain | Normal |
| 21 | 6p21.32 | 32,563,052 | 32,633,715 | 70,664 | Loss | Normal |
| 22 | 7q33 | 133,436,065 | 133,454,011 | 17,947 | Gain | Normal |
| 23 | 7q34 | 141,698,434 | 141,714,368 | 15,935 | Gain | Normal |
| 24 | 8p11.23, 8p11.22 | 39,352,161 | 39,505,456 | 153,296 | Gain | Normal |
| 25 | 8q24.23 | 137,751,705 | 137,922,791 | 171,087 | Loss | Normal |
| 26 | 10p12.1 | 27,646,417 | 27,746,073 | 99,657 | Loss | Normal |
| 27 | 11p15.4 | 4,926,383 | 4,932,414 | 6,032 | Gain | Normal |
| 28 | 11p15.4 | 5,742,276 | 5,765,638 | 23,363 | Gain | Normal |
| 29 | 11q11 | 55,123,519 | 55,209,826 | 86,308 | Loss | Normal |
| 30 | 12p13.2 | 11,121,004 | 11,140,621 | 19,618 | Loss | Normal |
| 31 | 14q21.1 | 40,680,389 | 40,727,130 | 46,742 | Loss | Normal |
| 32 | 14q24.3 | 73,071,204 | 73,092,312 | 21,109 | Gain | Normal |
| 33 | 14q32.33 | 105,080,369 | 106,035,030 | 954,662 | Gain | Normal |
| 34 | 14q32.33 | 106,222,937 | 106,252,326 | 29,390 | Loss | Normal |
| 35 | 16p11.1, 16p11.2 | 34,325,301 | 34,602,518 | 277,218 | Gain | Normal |
| 36 | 17q21.2 | 36,675,787 | 36,683,709 | 7,923 | Loss | Normal |
| 37 | 19q13.33 | 56,825,594 | 56,840,546 | 14,953 | Loss | Normal |
| 38 | 20p13 | 1,506,179 | 1,531,191 | 25,013 | Loss | Normal |
| 39 | 22q11.23 | 22,677,759 | 22,725,505 | 47,747 | Gain | Normal |

^{a}Classification based on Tsuchiya et al.²³

TABLE 6
CNVs detected in IV-3 with Agilent SurePrint G3 Human CGH 1×1M microarray

| Number | Cytoband | Start (Build 36) | Stop (Build 36) | Size (bp) | Type | Class^{a} |
|---|---|---|---|---|---|---|
| 1 | 19q13.33 | 55,872,843 | 55,934,778 | 61,936 | Loss | likely pathogenic |
| 2 | 7p15.3 | 21,468,768 | 21,479,251 | 10,484 | Loss | uncertain clinical significance |
| 3 | 1p36.21 | 12,769,321 | 12,840,191 | 70,871 | Loss | Normal |
| 4 | 1p31.1 | 72,533,604 | 72,579,511 | 45,908 | Gain | Normal |
| 5 | 1q21.3 | 150,822,873 | 150,851,639 | 28,767 | Loss | Normal |
| 6 | 2p13.2 | 73,706,527 | 73,764,697 | 58,171 | Gain | Normal |
| 7 | 2p11.2 | 88,913,881 | 88,941,277 | 27,397 | Gain | Normal |
| 8 | 2p11.2 | 88,944,777 | 89,093,846 | 149,070 | Gain | Normal |
| 9 | 3q26.1 | 164,009,121 | 164,027,924 | 18,804 | Loss | Normal |
| 10 | 4q13.2 | 69,069,451 | 69,166,014 | 96,564 | Loss | Normal |
| 11 | 5q35.3 | 180,344,764 | 180,366,177 | 21,414 | Loss | Normal |
| 12 | 5q35.3 | 180,447,092 | 180,465,652 | 18,561 | Loss | Normal |
| 13 | 6p21.33 | 30,021,708 | 30,031,567 | 9,860 | Loss | Normal |
| 14 | 7p21.3 | 8,793,643 | 8,830,093 | 36,451 | Loss | Normal |
| 15 | 7p14.1 | 38,270,742 | 38,360,387 | 89,646 | Loss | Normal |
| 16 | 7q31.1 | 109,230,136 | 109,240,410 | 10,275 | Gain | Normal |
| 17 | 10p12.1 | 27,646,417 | 27,746,073 | 99,657 | Loss | Normal |
| 18 | 11p15.4 | 5,738,523 | 5,766,644 | 28,122 | Gain | Normal |
| 19 | 11q11 | 55,118,014 | 55,220,185 | 102,172 | Gain | Normal |
| 20 | 12p13.31 | 9,528,390 | 9,610,254 | 81,865 | Loss | Normal |
| 21 | 14q11.2, 14q11.1 | 18,798,441 | 19,497,223 | 698,783 | Gain | Normal |
| 22 | 14q11.2 | 21,431,385 | 22,046,297 | 614,913 | Loss | Normal |
| 23 | 14q24.3 | 73,071,204 | 73,101,527 | 30,324 | Gain | Normal |
| 24 | 14q32.33 | 105,323,641 | 106,017,653 | 694,013 | Gain | Normal |
| 25 | 14q32.33 | 106,222,937 | 106,255,390 | 32,454 | Loss | Normal |
| 26 | 15q11.2 | 18,432,358 | 20,311,116 | 1,878,759 | Gain | Normal |
| 27 | 16q23.1 | 76,929,398 | 76,940,418 | 11,021 | Gain | Normal |
| 28 | 17q21.2 | 36,675,787 | 36,683,709 | 7,923 | Loss | Normal |
| 29 | 20p13 | 1,511,432 | 1,532,633 | 21,202 | Loss | Normal |
| 30 | 21q11.2 | 13,825,429 | 14,125,379 | 299,951 | Gain | Normal |
| 31 | 22q11.23 | 22,677,759 | 22,725,505 | 47,747 | Loss | Normal |
| 32 | Xq12 | 65,684,735 | 65,848,843 | 164,109 | Gain | Normal |

^{a}Classification based on Tsuchiya et al.²³

### SHANK1 Sequencing in ASD and ID

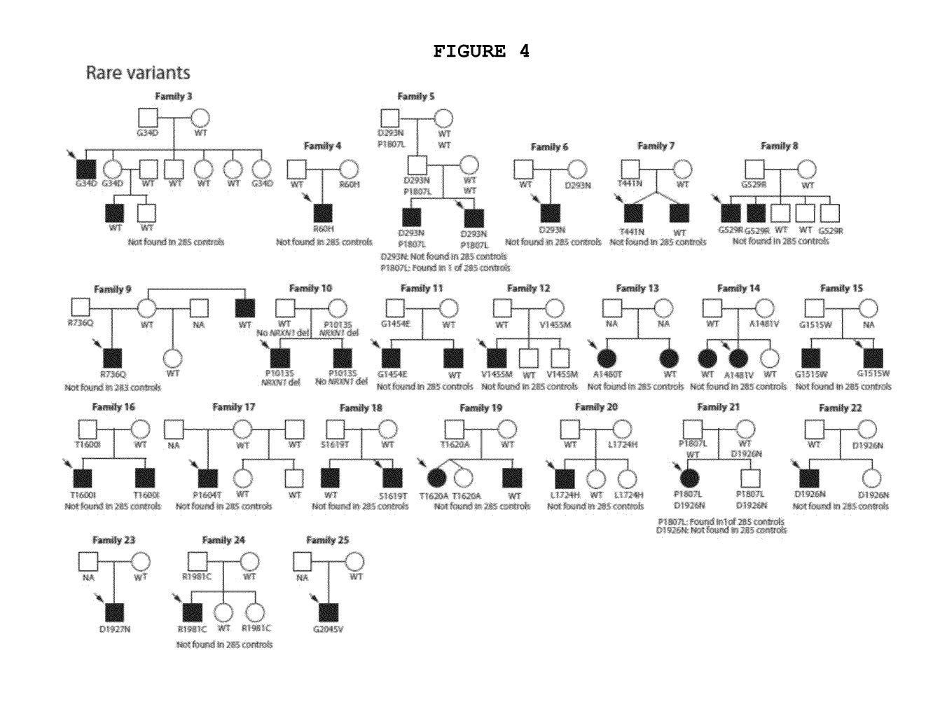

To test for sequence-level mutations in SHANK1, Sanger sequencing was used to examine all 23 exons and splice sites in 509 unrelated ASD (384 male and 125 female) and 340 ID (191 males and 149 females). Detected were 26 rare missense variants in 23 ASD and 7 ID cases, which were not found in the Single Nucleotide Polymorphism Database (dbSNP) build 130 or in 285 control individuals from the Ontario general population (as shown in Table 7 below and FIG. 4). Two of these missense variants (D293N in Families 5 and 6 and R736Q in Family 9) are predicted to be damaging based on their alteration of highly conserved residues within the ANK and PDZ domains, respectively. While they occur in males with ASD, both variants are also found in non-ASD fathers. No significant mutation was found on the non-deleted allele of the proband III-5.

TABLE 7
Missense variants found at SHANK1 locus in ASD and ID patients.

| | Nucleotide | AminoAcid | | | | | Occurrence | | |
|---|---|---|---|---|---|---|---|---|---|
| Individual | Change | Change | Conservation | Gender | Exon | Inheritance | ASD | ID | Controls |

Rare missense variants in ASD probands (absent in controls)

| | | | | | | | | | |
|---|---|---|---|---|---|---|---|---|---|
| Family 3 | c.101 G > A | G34D | 0 | Male | 1 | Paternal | 1 | 0 | 0 |
| Family 4 | c.179 G > A | R60H | 0 | Male | 1 | Maternal | 1 | 0 | 0 |
| Family 5 | c.877 G > A | D293N | 3 | Male | 6, ANK domain | Paternal | 2 | 0 | 0 |
| Family 6 | | | | Male | | Maternal | | | |
| Family 7 | c.1322 C > A | T441N | 0 | Male | 10 | Paternal | 1 | 1 | 0 |
| Family 8 | c.1585 G > A | G529R | 0 | Male | 11 | Paternal | 1 | 0 | 0 |
| Family 9 | c.2207 G > A | R736Q | 2 | Male | 17, PDZ domain | Paternal | 1 | 0 | 0 |
| Family 10 | c.3037 C > T | P1013S | 0 | Female | 22 | Maternal | 1 | 0 | NT |
| Family 11 | c.4361 G > A | G1454E | 0 | Male | 22 | Paternal | 1 | 0 | 0 |
| Family 12 | c.4363 G > A | V1455M | 0 | Male | 22 | Maternal | 1 | 0 | 0 |
| Family 13 | c.4438 G > A | A1480T | 0 | Female | 22 | ND | 1 | 0 | 0 |
| Family 14 | c.4442 C > T | A1481V | 0 | Female | 22 | Maternal | 1 | 0 | 0 |
| Family 15 | c.4543 G > T | G1515W | 0 | Male | 22 | Paternal | 1 | 0 | 0 |
| Family 16 | c.4799 C > T | T1600I | 0 | Male | 22 | Paternal | 1 | 0 | 0 |
| Family 17 | c.4810 C > A | P1604T | 0 | Male | 22 | ND | 1 | 0 | 0 |
| Family 18 | c.4855 T > A | S1619T | 0 | Male | 22 | Paternal | 1 | 0 | 0 |
| Family 19 | c.4858 A > G | T1620A | 0 | Female | 22 | Paternal | 1 | 0 | 0 |
| Family 20 | c.5171 T > A | L1724H | 0 | Male | 22 | Maternal | 1 | 0 | 0 |
| Family 21 | c.5776 G > A | D1926N | 0 | Female | 23 | Maternal | 2 | 0 | 0 |
| Family 22 | | | | Male | | Maternal | | | |
| Family 23 | c.5779 G > A | D1927N | 0 | Male | 23 | ND | 1 | 0 | NT |
| Family 24 | c.5941 C > T | R1981C | 0 | Male | 23 | Paternal | 1 | 1 | 0 |
| Family 25 | c.6134 G > T | G2045V | 0 | Male | 23 | ND | 1 | 0 | NT |

Missense variants present in cases and controls

| | | | | | | | | | |
|---|---|---|---|---|---|---|---|---|---|
| Family 26 | c.3947 G > A | G1316D | 0 | Female | 22 | Paternal | 2 | 0 | 3 |
| Family 27 | | | | Male | | ND | | | |
| Family 28 | c.5387 G > A | G1796E | 0 | Male | 22 | Maternal | 7 | 1 | 5 |
| Family 29 | | | | Male | | Paternal | | | |
| Family 30 | | | | Female | | ND | | | |
| Family 31 | | | | Male | | Maternal | | | |
| Family 32 | | | | Female | | Maternal | | | |
| Family 33 | | | | Male | | ND | | | |
| Family 34 | | | | Male | | Maternal | | | |
| Family 5 | c.5420 C > T | P1807L | 0 | Male | 22 | Paternal | 3 | 5 | 1 |
| Family 21 | | | | Female | | Paternal | | | |
| Family 35 | | | | Male | | Paternal | | | |

Rare missense variants in ID cases

| | | | | | | | | | |
|---|---|---|---|---|---|---|---|---|---|
| MR44 | c.1322 C > A | T441N | 0 | Female | 10 | Paternal | 1 | 1 | 0 |
| S03445 | c.2534 C > A | A845E | 0 | Female | 20 | Unknown | 0 | 1 | 2 |
| MR81 | c.2629 T > A | F877I | 0 | Male | 21 | Paternal | 0 | 1 | 0 |
| S03455 | c.3629 C > A | S1210Y | 0 | Male | 22 | Maternal | 0 | 1 | NT |
| MR66 | c.5305 C > T | R1769W | 0 | Female | 22 | Paternal | 0 | 1 | NT |
| MR9 | c.5387 G > A | G1796E | 0 | Male | 22 | Maternal | 7 | 1 | 5 |
| MR3 | c.5420 C > T | P1807L | 0 | Female | 22 | Maternal | 3 | 5 | 1 |
| MR45 | | | | Male | | Maternal | | | |
| MR179 | | | | Female | | ND | | | |
| MR219 | | | | Female | | Paternal | | | |
| MR224 | | | | Female | | Paternal | | | |
| S03489 | c.5531 C > G | P1844R | 0 | Male | 22 | Paternal | 0 | 1 | 0 |
| MR210 | c.5732 A > G | Y1911C | 0 | Male | 22 | Paternal | 0 | 1 | 0 |
| MR55 | c.5941 C > T | R1981C | 0 | Female | 23 | Paternal | 1 | 1 | 0 |

Proband from Family 10 has a 17-kb (50, 704, 743-50, 721, 920) (hg18) maternally transmitted deletion at 2p16.3 (Validated and mapped, data not shown) disrupting one exon of NRXN1. This Individual has Indian origin. Proband from Family 3 has balanced translocation t (3; 15) (q26.2; q21.2).
SHANK1 Conservation (Amino acid is conserved in all SHANK genes (3), in SHANK1 and in one if either SHANK2 or SHANK3 (2) or not conserved in any of the SHANK genes (0)). The total number of individuals sequence is 509 ASD (Male 384, Female 125), 340 ID (Males 191, Females 149) and TaqMan testing was done for 285 control individuals (138 Males and 147 Females). ND, not determined; NT not tested.

### Genome Sequencing and Analysis in Family 1

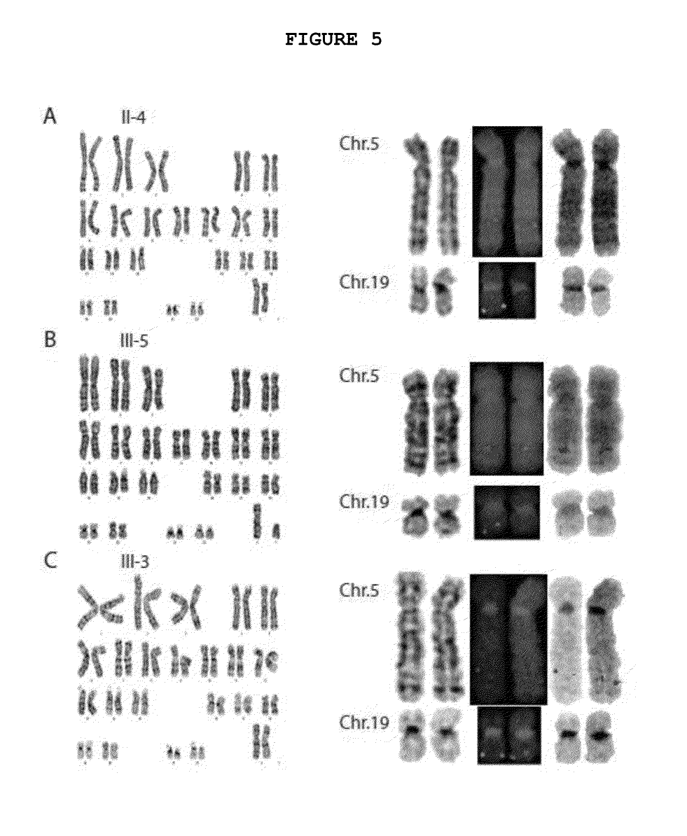

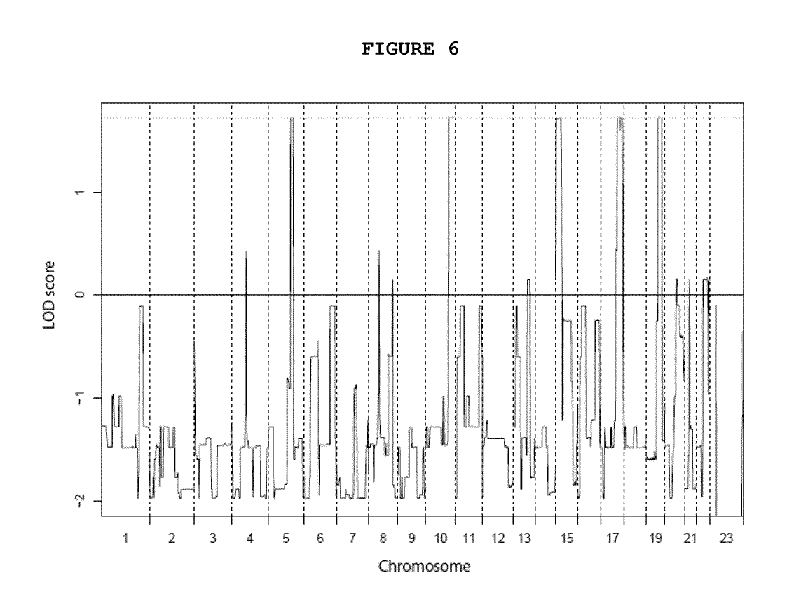

Whole-exome sequencing was conducted in subjects III-5 and IV-3 from Family 1 to search for potential mutations in other genes (see Table 4 below of the Supplementary Appendix). A non-sense mutation predicted to introduce a stop codon (Y313X) in the PCDHGA11 prodocadherin gene on chromosome 5q31.3 was identified. PCDHGA11 is a member of the protocadherin gamma gene cluster thought to have an important role in establishing connections in the brain. The mutation was found to segregate precisely with the SHANK1 deletion. Since SHANK1 and PCDHGA11 reside on different autosomes, translocation or transposition was tested for, and such linkage was ruled out (FIG. 5). It is possible that the Y313X mutation in PCDHGA11 works in concert with the SHANK1 deletion to modify (positively or negatively) the extent of the phenotype or that they are just randomly co-segregating; however, CNV or sequence-level mutations in PCDHGA11 in Family 2 or in any other ASD subject examined were found. The role of the X chromosome in Family 1 has been ruled out given different X-chromosomes were observed in ASD males (based on comparison of SNP genotypes), and no pathogenic CNV, mutation or genetic linkage was observed at the X chromosome (FIG. 6).

TABLE 8
SNVs detected by exome sequencing ASD cases
with SHANK1 deletions

| | | | | | Exonic | Exonic |
|---|---|---|---|---|---|---|
| | Alignment | | | | novel non- | novel |
| | with | | | Exonic | synon- | synon- |
| | HuRefZ* | Total | Exonic | novel | ymous | ymous |
| Sample | (%) | SNVs | SNVs | SNVs | SNVs | SNVs |
| III-5 | 93.3 | 52,310 | 17,105 | 1,644 | 994 | 590 |
| IV-3 | 92.4 | 133,959 | 40,956 | 20,724 | 14,360 | 5,447 |

*HuRef, human reference genome NCBI Build 37/hg19

### Analysis of ASD Individuals and Families

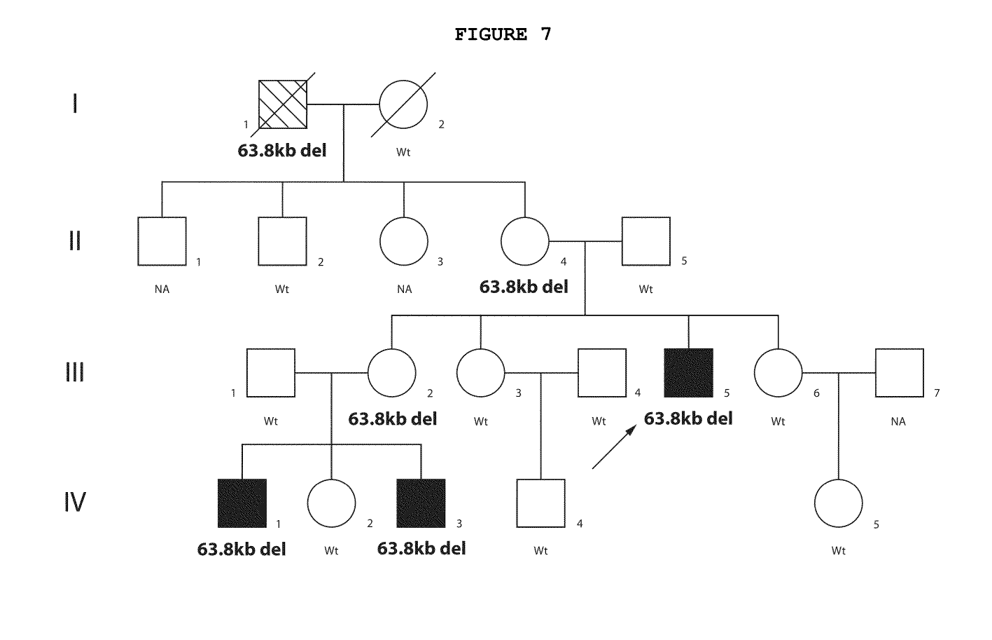

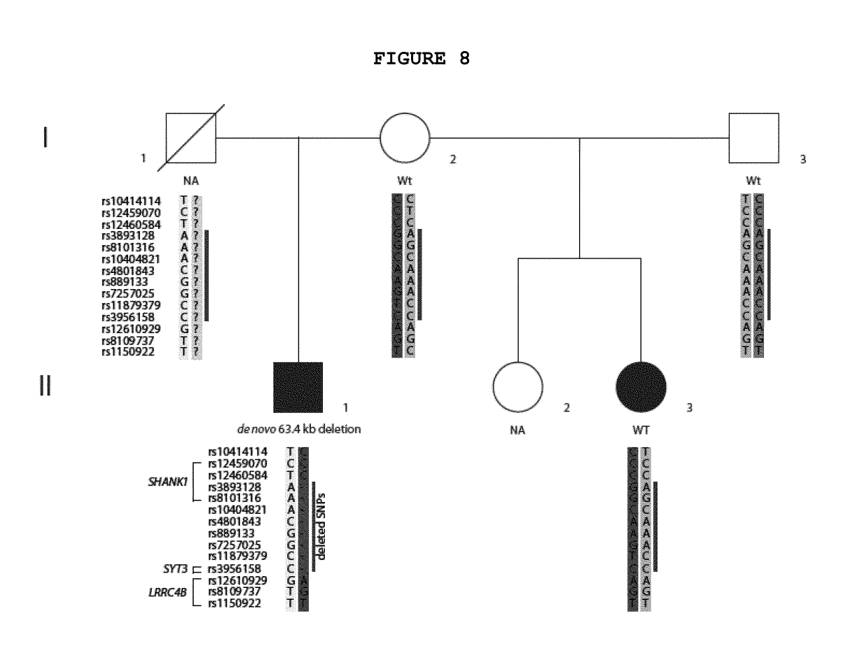

Individuals with deletions involving SHANK1, including four male cases with higher-functioning ASD or the BAP from a multi-generation family carrying inherited gene deletions (FIG. 7), and an unrelated fifth ASD male case with a de novo deletion at the same locus (FIG. 8) were revealed in this study and are described in detail below.

### Family 1:

The proband III-5 from family 1 (FIG. 7 and Table 9) was first assessed by a child psychiatrist at age 16 and was initially given a clinical diagnosis of PDD-NOS. There was evidence of impairment in social-communication starting at an early age, but not enough repetitive stereotyped behaviors for a diagnosis of autism or Asperger disorder. An Autism Diagnostic Interview Revised (ADI-R) and an Autism Diagnostic Observational Survey (ADOS) were completed at age 25. The ADI-R indicated that the parents first became concerned in the 12-24 month period, when III-5 engaged in repetitive play and speech. He spoke in single words at 24 months, and in phrases by 36 months. There has never been a loss of language or of other skills. There was no history of echolalia, pronoun reversal or neologisms. His eye contact was always poor, and there has been a persistent lack of social smiling, facial affect, joint attention and empathy. His interests over childhood and adolescence included video games, movies and sports cards. He graduated from high school and now at age 32 lives independently and works in a sheltered workshop. His current best-estimate diagnoses are that of Asperger disorder and an anxiety disorder. An extensive battery of questionnaires and tests were administered to III-5's parents and both scored in the typical range. His mother (II-4) has exhibited anxiety and shyness most of her life, but would not be considered BAP. His 40 year-old sister (III-2) is married with one son (IV-1) with Asperger disorder, a neurotypical daughter (IV-2), and a son (IV-3) with ASD. III-2 completed university and worked as a school teacher for years. She has a diagnosis of social anxiety and generalized anxiety disorder for which she has taken anti-anxiety medication. Assessment by interview and questionnaire indicated she was typical for all measures and did not show evidence of the BAP.

IV-1 was clinically diagnosed with Asperger disorder at age 8. He was born 10 days overdue by cesarean section. Early developmental milestones were within normal limits. His parents appreciated developmental differences at age 3 when it was noted that he was not interested in other children and was preoccupied with objects. He had an encyclopedic knowledge of cars. He would approach other children, but tended to play beside them and became upset with changes in routine. He exhibited difficulties with eye contact and understanding social cues and rules. Additional assessment was conducted at age 10. He met all the cut-offs for autistic disorder on the ADI-R except for the nonverbal total. The ADOS, scores were below cut-off for a diagnosis of ASD due to strengths in the communication domain. Descriptive gestures, were present, although they were vague and infrequent, accounting for his communication score of 1 (cut-off is 2). Impairments in reciprocal social interaction continued to be evident. On psychometric testing, he had a significant verbal-performance discrepancy with lower performance than verbal scores (114 vs. 86). IV-1 qualified for a diagnosis of Asperger disorder.

Individual IV-3 was first evaluated at age 3. At 18 months, his parents became concerned because he was not talking He developed single words at 24 months. He communicated by leading his parents by the hand and exhibited repetitive behaviors. He did not offer comfort or empathy and did not initiate social interaction, although he would play with his parents. Certain noises bothered him such as the washing machine or the toilet flushing; he became upset if his mother had her hair down or a jacket unzipped. Assessment at age 5 years 8 months indicated he was positive on the ADI-R for autism and for ASD on the ADOS. He had made good progress in social interaction and language. His expressive language consisted of short sentences and phrases with some echoed speech and mild articulation difficulties. His IQ and expressive and receptive language scores were in the low average range, leading to a best-estimate diagnosis of Asperger disorder.

TABLE 9
Clinical description of individuals carrying SHANK1 deletion

| Family | Clinical Details |
|---|---|
| Family 1 | |
| III-5 (male) | Dx^{a}: ASD: Asperger disorder (ADI-R & ADOS-4) and anxiety; IQ^{b}: (Leiter-R) Brief |
| | NVIQ = 83 (13% ile)/LA; Language^{c}: (OWLS) TL = 68 (2% ile)/Delay; Adaptive |
| | Behaviour^{d}: (VABS-I) ABC = 52 (<1% ile), COM = 43 (<1% ile), DLS = 63 (1% ile), |
| | SOC = 65 (1% ile). He currently takes olanzapine and paroxetine for the anxiety disorder. |
| I-1 (male) | Dx: Broader autism phenotype. Shy/reserved and reluctant to approach people. Amassed a |
| | large stamp collection. Deceased. |
| IV-1 (male) | Dx: ASD: Asperger disorder (ADI-R), SRS: 68T/Mild-Moderate; IQ: (WASI) VIQ = 114 |
| | (82% ile)/HA > PIQ = 86 (18% ile)/LA; Language: (OWLS) TL = 93, RL = 82 (12% ile), |
| | EL = 107 (68% ile), PPVT RV = 97 (42% ile); Adaptive Behaviour: (VABS-II) ABC = 85 |
| | (16% ile), COM = 92 (30% ile), DLS = 85 (16% ile), SOC = 85 (16% ile). |
| IV-3 (male) | Dx: ASD: Asperger disorder (ADI-R & ADOS-3); IQ: (WPPSI) FSIQ = 89 (23% ile)/LA, |
| | VIQ = 89 (23% ile), PIQ = 91 (27% ile); Language: (OWLS) TL = 80 (9% ile), RL = 78 (7% |
| | ile), EL = 86 (18% ile), (PPVT) RV = 91 (27% ile); Adaptive Behaviour: (VABS-II) |
| | ABC = 86 (18% ile), COM = 91 (27% ile), DLS = 89 (23% ile), SOC = 86 (16% ile), MOT = 91 |
| | (27% ile). |
| II-4 (female) | Dx: Non-ASD. Anxiety and Shyness. |
| III-2 (female) | Dx: Non-ASD. Social Anxiety Disorder and Generalized Anxiety Disorder. Shy as a child. |
| | Language: (PPVT) RV = 111 (77% ile). |
| Family 2 | |
| II-1 (male) | Dx: ASD, high functioning (ADI-R; CARS: mild autism); IQ: (WISC) FSIQ = 115 (84% |
| | ile)/HA, VIQ = 120 (93% ile), PIQ = 100 (50% ile), (VIQ > PIQ); Brain imaging (PET): mild |
| | hyperfusion temporal left. |

Refer to pedigrees in Fig. 7(Family 1) and Fig. 8 (Family 2).
Abbreviations used: ASD: Autism Spectrum Disorder; PET: Positron Emission Tomography.
^{a}Autism Spectrum Diagnosis based on Autism Diagnostic Interview-Revised (ADI-R) and Autism Diagnostic Observation Schedule (ADOS; one of 4 possible modules administered based on age and language level). In some cases the Social Responsiveness Scale (SRS) was administered and reported T-scores represent Average skills (≦59T), Mild to Moderate Concerns (60T to 75T), Severe range (76T or higher). Also the diagnosis for II-1 in Family 2 was based on the Childhood Autism Rating Scale (CARS).
^{b}IQ measured using age appropriate Weschler scale (WPPSI-Wechsler Preschool and Primary Scale of Intelligence; WISC-Intelligence Scale for Children; WASI-Wechsler Abbreviated Scale of Intelligence). Standard scores and percentiles (% ile) presented for full scale IQ (FSIQ), verbal IQ (VIQ) and/or performance IQ (PIQ). FSIQ is not a valid estimate of IQ when significant discrepancy exists between VIQ and PIQ. Leiter International Performance Scale-Revised (Leiter-R) is a measure of non-verbal IQ (NVIQ) only. Percentile classifications: Very Superior (VS; >98^{th} % ile), Superior (S; 91^{st}-97^{th} % ile), High Average (HA; 75^{th}-90^{th} % ile), Average (A; 25^{th}-74^{th} % ile), Low Average (LA; 9^{th}-24^{th} % ile), Borderline (B; 2^{nd}-8^{th} % ile), and Extremely Low (EL; <2^{nd} % ile).
^{c}Language measured using the Oral and Written Language Scales (OWLS). Standard scores and percentiles presented for total language (TL), receptive language (RL), and/or expressive language (EL). Language was rated as nonverbal, average, or delayed (≦16^{th} % ile). The Peabody Picture Vocabulary Test (PPVT-4^{th} edition) measured receptive vocabulary (RV).
^{d}Adaptive Behavior measured using the Vineland Adaptive Behavior Scales (VABS which edition. Standard score and percentiles presented for Adaptive Behavior Composite (ABC); Communication (COM); Daily Living Skills (DLS); Socialization (SOC); Motor (MOT; only for children aged 7 years or less).

### Family 2:

Male individual F2-II-1 (FIG. 8 and Table 9) was the first child born to a 20-year old mother. He has a younger maternal half-sister (F2-II-3) with autism and mild ID. F2-II-1 was born two months before term. Developmental abnormalities were identified during his first year. He did not babble, made no eye contact, and refused to be touched. He started to walk at age 2, but motor coordination was poor. He started to talk at age 2.5 years, which astonished the parents because until then he had been extremely quiet. He developed a formal, pedantic style of speech with abnormal prosody. He was uninterested in other children. He repeated routines and rituals and accumulated facts on certain subjects such as astronomy. When upset, he flapped his hands or moved his body in a stereotypic fashion. Lately, he had periods of depression. His IQ was in the normal range with good verbal ability. The best estimate diagnosis was high-functioning autism.

### Discussion

This is the first description of hemizygous deletions of the SHANK1 gene in ASD. The striking segregation of ASD in only male SHANK1 deletion carriers in Family 1 represents the first example of autosomal sex-limited expression in ASD. The finding of an unrelated male with ASD carrying an independent de novo deletion of SHANK/supports the interpretation that the SHANK1 CNV segregating in Family 1 is indeed the primary etiologic event leading to ASD.

The data indicate SHANK1 deletions are associated with higher-functioning ASD in males. Insofar as all affected males have IQ in the typical range and have good verbal ability (with a lack of clinically significant language delay), they would also qualifiy for a diagnosis of Asperger disorder.

It is noted that the neuronal genes PCDHGA11 and SYT3, could also contribute to aspects of the ASD phenotype in Family 1 and Family 2, respectively.

We claim:

1. A method of assessing risk in a human subject of ASD comprising the step of identifying in a nucleic acid-containing sample obtained from the human subject copy number variations associated with SHANK1, wherein a determination of copy number variations associated with SHANK1 is indicative of a risk of ASD in the human subject.

2. The method of claim 1, wherein the CNV is a deletion.

3. A method of assessing risk in a human subject of ASD comprising the step of identifying in a protein acid-containing sample obtained from the human subject the expression or activity of a SHANK1 protein product, comparing the expression or activity of said protein product with the normal expression or activity of said product, wherein a determination of an expression or activity of said product that is different from said normal expression or activity is indicative of a risk of ASD in the human subject.


SEQUENCE LISTING (94 sequences)

SEQ ID NO: 1 - DNA, 6643 bp, Homo sapiens
gtcgccccgtggccccacaatgacccacagccccgcgacaagcgaggacgaggaacgccacagtgccagcgagtgtcccgaggggggctcagagtccgacagctccccagacgggccaggtcgaggcccccgggggacccggggccagggcagtggggcacctggtagcctggcctctgttagaggcctccagggccgctcaatgtccgtcccagacgacgcccacttcagcatgatggtcttcaggattggcatcccggacctgcaccagacaaaatgccttcgcttcaaccccgatgccaccatctggacggccaagcagcaggtgctctgtgccctgagcgagagcctgcaggatgtgctcaactatggcctgttccaaccggccacctccggccgcgatgccaacttcctggaggaggagaggctgctgcgggagtacccccagtcctttgagaagggggtcccctacctggagttccgatacaagacccgagtttacaaacagaccaacctggatgagaagcagctggccaagttgcacacgaagacggggttgaagaagttcctggagtatgtgcagctcgggacatctgacaaggtggcgcggctgctggacaaggggctggaccccaattaccatgactcggattcgggagagacccccttgacactggcggcccagaccgaaggctctgtagaggtgattcgaaccctgtgcctgggcggggcccacattgacttccgggcccgggatggcatgaccgcactgcataaggccgcatgcgcccgacactgcctggcactcacggcgctcctggaccttgggggttcccccaactacaaggaccgtcgggggctgacccctctgttccacacggccatggtgggtggtgacccccgatgctgcgagctgctcctgttcaacagggcccagctgggcatagctgatgagaacggctggcaggaaatccaccaggcctgccagcggggtcactctcagcacctggagcatctgcttttctacggggctgagcctggagcccagaacgcctcggggaacacggctctgcacatctgcgccctctacaacaaggagacctgtgccaggatcctcctgtatcgaggtgccgacaaggatgtgaagaacaacaacggacagacccccttccaggtggcagtgattgctgggaattttgagctgggggagctgatccgaaaccaccgagaacaggatgtggtgcccttccaggagtcccccaagtacgcggcccggcgacgggggcccccaggcacagggctgacggtgcccccggcgctgctgcgggccaacagtgacaccagcatggcgctgcccgactggatggtgttctccgccccgggggccgcgtcctctggggcccctggccctacctcagggtcccagggccagtcgcagccctcggcccccaccaccaagctcagcagcgggaccctccgaagtgccagcagcccccggggtgccagggcccgctctccatcccgagggaggcaccctgaggacgccaagaggcagccccgaggccggcccagctccagcgggacaccccgggaagggccagccgggggcacggggggctcagggggccccgggggctccctgggcagccgcgggaggcggaggaagctctactcagcggtacccggacgctccttcatggctgtgaagtcctaccaggcccaagccgagggggagatctccctgagcaagggcgagaagatcaaagtacttagcatcggggaaggaggcttctgggaaggccaggtcaaaggtcgtgttggctggttcccctctgactgcctggaagaagtggcgaatcgctctcaggagagcaagcaagaaagccgcagtgacaaggcaaagagactcttccggcattataccgtgggctcctacgacagctttgatgccccaagcttaatggatgggattggcccagggagcgattacatcattaaggagaagacagtcttgctgcagaagaaggacagtgaggggtttgggttcgtgctccggggggccaaggcgcagacccccatcgaggagttcacccccaccccggccttcccggcgctgcagtacctggagtcggtggacgagggtggcgtggcatggcgagctggactgcgaatgggagacttcctcatcgaggtgaacgggcagaatgtggtgaaggtcggccaccgacaggtggtgaacatgatccgccaagggggcaacacgctgatggtgaaggtggtgatggtcaccaggcacccggacatggatgaggcagtgcacaagaaagcaccccagcaggccaagcggctgccgcccccaaccatctccctgcgttccaaatctatgacctcagagctggaggagatggagtacgagcagcagccggcgccggtgcccagcatggagaaaaagcggaccgtgtatcagatggctctcaacaaactggacgaaatcctggccgcagctcaacagaccatcagtgcaagcgaaagccctggtcccggtggcctcgcgtccctgggcaaacaccgacccaaaggtttctttgccactgagtcgagcttcgatccccaccaccgtgcccagccaagttacgagcgtccttctttcctgcctccaggacctgggttgatgctccggcaaaaatctatcggtgcggcagaagatgacagaccttacctagcacccccagccatgaaattcagccgcagcctgtctgtgcctggttcggaggacattcccccgccacccaccacgtccccaccggagcctccctacagcacacctccagtcccctcctcctcagggcgcctcaccccctcccctcggggagggcccttcaaccctggctctggtggccccctccccgcctcctcccctgcatcctttgacgggccctcccctcccgacactcgcgtggggagccgcgagaagagcctgtaccacagtgggcccctgcccccggcccaccaccacccgccccaccaccaccaccaccacgccccgccccctcagccccaccaccaccacgcccacccccctcatcctcccgagatggagacaggcggctctcccgacgaccctccaccccgcctggctctggggccccagcccagcctgcgaggctggaggggcggcgggcccagcccgaccccgggggccccgtccccatcgcaccacggcagcgcgggcgggggcggcggctcctcccagggcccggctctacgctatttccagctgcccccgcgggcggccagcgcagccatgtacgtgcccgcccgctcgggccgcggccgcaagggcccgctggtcaagcagaccaaggtggaaggcgagccccagaagggcggcggcctcccgcccgcgccgtcgcccacgtccccggcctccccgcagccgccgcccgccgtggccgcgccctcggagaagaactccatccccatccccaccatcatcatcaaggccccgtccaccagtagcagcggccgcagcagccagggcagcagcaccgaggcggagccccccacccagccggagcccacgggaggcggcggcggcggcggctcctcgcccagccccgccccggccatgtcacccgtgcccccgtccccctcgcccgtgcccacccccgcctcgcccagcggcccggccacgctggacttcacgagccagttcggggccgccctggtgggggcggcccggagggaggggggctggcagaatgaggcgcgccggcgctccacgctgttcctgtccaccgacgcgggggacgaggacggcggggacggcgggctgggcacaggggcggccccgggcccgcggctgcgccactccaaatccatcgacgagggcatgttctccgccgagccctacctccgactggagtctgcgggcagcggcgcgggctacggcggctacggggccggtagccgagcctacgggggtggcgggggcagcagcgccttcaccagcttcctgcccccgcgacccctggtgcacccgctgaccggcaaggccctggatcccgcctccccgctggggctggccctggccgcccgcgagcgagcgctgaaggagtcctcggagggcggcggggccccccagccgcctcccaggcccccatcgccccgctacgaggccccgccgcccaccccgcaccaccactcgccccacgcccaccacgagccagtgctgcgtctctggggggcctccccgccggaccctgcgcgccgggagctggggtacagggccgggctgggcagccaggagaagtcccttcccgccagcccgcccgccgcccggcgttccctgctacaccgcctgccgcccaccgctcccggggtggggcccctcctgctgcagctggggacggagcccccggccccgcaccccggagtaagcaagccctggaggtccgcagcccccgaagaacccgagcggctgccgctgcacgtgcggttccttgaaaactgccagccccgggcccctgtgacgagcggaaggggtcccccctcggaggacgggccgggggtcccgccgcccagcccacgccggtccgtgcccccctccccgacctccccgagggccagcgaagagaacgggctgcccctgctggtcctgccgcctcccgccccctcggtggatgtggaagatggcgaattccttttcgtggaaccgctgcctccgcctctggaattctccaacagcttcgaaaagccagagtcgcccctcacgcctgggcctccccacccgctgcccgacacacctgcccctgccaccccgttaccccctgtgccacccccggctgtggccgcagcccctcccaccctggactccaccgcatccagcctgacatcctatgacagcgaggtggccaccctgacccagggggcctccgccgctcctggggacccccatccaccaggcccgcctgccccagcagcaccggctcccgctgccccacagcctggcccggaccctccgcctggcacggattctggcatcgaggaggtggacagtcggagcagcagtgaccacccactggagaccatcagcagcgcctccacgctgagcagcctatctgccgaaggtggtggcagcgcagggggtgggggcggggctggggccggtgtggccagtgggccggagcttctggacacctatgtggcctacctggacggccaggcctttgggggcagcagtactcccggcccgccataccctcctcagctcatgactccctctaagctccggggccgggcgctaggagccagcggaggcctgcggcctggccccagcgggggactccgagaccctgttacccccaccagccccaccgtctcggtgacaggggctggaaccgatgggctgctggccctgcgtgcttgttcaggaccccccacggcaggcgtggcggggggtccggtggctgtagagccagaagtcccaccggtgcccttgccgacggcctcctctctgccccggaagctgctgccctgggaggagggcccgggcccaccgccaccacctctgcccgggcccttggcccagcctcaggcctcagccttggccacagtaaaagccagcatcatcagtgaactcagctccaagcttcagcagtttgggggctcctcggcagctggcggcgctctgccctgggcccgaggaggcagtgggggaggcggagacagccaccacgggggagccagctatgtccccgagaggacctcctccctgcagcggcagagactctccgacgactcccagtcctcactcctctccaagcctgtcagcagcctgtttcagaactggcccaaaccacctctgccgccactccccaccggaacaggggtctcccctacagccgctgcggccccaggggccacctcaccctcagcctcctcctcctccacgtccacccgccacctccagggcgtggagttcgagatgcggccccctctgctccgccgggcccccagcccctcgctgctgcccgcctcggagcacaaggtcagccctgcgcccaggccctcgtccctgcccatcctgccttccggacccctctacccaggcctctttgacatccgtggctccccaactggaggggcaggaggctcggctgaccccttcgccccagtctttgtgccgccacacccggggatatccggggggctcgggggagccttgtcaggggcctcgcgctccctctcaccgacccgcctgctctcgctgcccccggacaagccgtttggcgctaaacctctggggttctggaccaagttcgacgtggctgattggctggagtggctgggtttggcggagcaccgagcccagttcctggaccacgagatcgatggctcccacctgcccgccttgaccaaggaggactacgtcgatctaggtgtgaccagggtgggccaccgcatgaacatcgaccgggctctcaaattcttcctggagaggtgatggctggcctggacggaccagccccgtccacagaactcttgagcctgctggcctcttgacctctgacccctgactgtcattctctccccgggccagggactctgttcaaactgcgccctgccctcatctcccaaggcc

SEQ ID NO: 2 - protein, 2160 aa, Homo sapiens
MTHSPATSEDEERHSASECPEGGSESDSSPDGPGRGPRGTRGQGSGAPGSLASVRGLQGRSMSVPDDAHFSMMVFRIGIPDLHQTKCLRFNPDATIWTAKQQVLCALSESLQDVLNYGLFQPATSGRDANFLEEERLLREYPQSFEKGVPYLEFRYKTRVYKQTNLDEKQLAKLHTKTGLKKFLEYVQLGTSDKVARLLDKGLDPNYHDSDSGETPLTLAAQTEGSVEVIRTLCLGGAHIDFRARDGMTALHKAACARHCLALTALLDLGGSPNYKDRRGLTPLFHTAMVGGDPRCCELLLFNRAQLGIADENGWQEIHQACQRGHSQHLEHLLFYGAEPGAQNASGNTALHICALYNKETCARILLYRGADKDVKNNNGQTPFQVAVIAGNFELGELIRNHREQDVVPFQESPKYAARRRGPPGTGLTVPPALLRANSDTSMALPDWMVFSAPGAASSGAPGPTSGSQGQSQPSAPTTKLSSGTLRSASSPRGARARSPSRGRHPEDAKRQPRGRPSSSGTPREGPAGGTGGSGGPGGSLGSRGRRRKLYSAVPGRSFMAVKSYQAQAEGEISLSKGEKIKVLSIGEGGFWEGQVKGRVGWFPSDCLEEVANRSQESKQESRSDKAKRLFRHYTVGSYDSFDAPSLMDGIGPGSDYIIKEKTVLLQKKDSEGFGFVLRGAKAQTPIEEFTPTPAFPALQYLESVDEGGVAWRAGLRMGDFLIEVNGQNVVKVGHRQVVNMIRQGGNTLMVKVVMVTRHPDMDEAVHKKAPQQAKRLPPPTISLRSKSMTSELEEMEYEQQPAPVPSMEKKRTVYQMALNKLDEILAAAQQTISASESPGPGGLASLGKHRPKGFFATESSFDPHHRAQPSYERPSFLPPGPGLMLRQKSIGAAEDDRPYLAPPAMKFSRSLSVPGSEDIPPPPTTSPPEPPYSTPPVPSSSGRLTPSPRGGPFNPGSGGPLPASSPASFDGPSPPDTRVGSREKSLYHSGPLPPAHHHPPHHHHHHAPPPQPHHHHAHPPHPPEMETGGSPDDPPPRLALGPQPSLRGWRGGGPSPTPGAPSPSHHGSAGGGGGSSQGPALRYFQLPPRAASAAMYVPARSGRGRKGPLVKQTKVEGEPQKGGGLPPAPSPTSPASPQPPPAVAAPSEKNSIPIPTIIIKAPSTSSSGRSSQGSSTEAEPPTQPEPTGGGGGGGSSPSPAPAMSPVPPSPSPVPTPASPSGPATLDFTSQFGAALVGAARREGGWQNEARRRSTLFLSTDAGDEDGGDGGLGTGAAPGPRLRHSKSIDEGMFSAEPYLRLESAGSGAGYGGYGAGSRAYGGGGGSSAFTSFLPPRPLVHPLTGKALDPASPLGLALAARERALKESSEGGGAPQPPPRPPSPRYEAPPPTPHHHSPHAHHEPVLRLWGASPPDPARRELGYRAGLGSQEKSLPASPPAARRSLLHRLPPTAPGVGPLLLQLGTEPPAPHPGVSKPWRSAAPEEPERLPLHVRFLENCQPRAPVTSGRGPPSEDGPGVPPPSPRRSVPPSPTSPRASEENGLPLLVLPPPAPSVDVEDGEFLFVEPLPPPLEFSNSFEKPESPLTPGPPHPLPDTPAPATPLPPVPPPAVAAAPPTLDSTASSLTSYDSEVATLTQGASAAPGDPHPPGPPAPAAPAPAAPQPGPDPPPGTDSGIEEVDSRSSSDHPLETISSASTLSSLSAEGGGSAGGGGGAGAGVASGPELLDTYVAYLDGQAFGGSSTPGPPYPPQLMTPSKLRGRALGASGGLRPGPSGGLRDPVTPTSPTVSVTGAGTDGLLALRACSGPPTAGVAGGPVAVEPEVPPVPLPTASSLPRKLLPWEEGPGPPPPPLPGPLAQPQASALATVKASIISELSSKLQQFGGSSAAGGALPWARGGSGGGGDSHHGGASYVPERTSSLQRQRLSDDSQSSLLSKPVSSLFQNWPKPPLPPLPTGTGVSPTAAAAPGATSPSASSSSTSTRHLQGVEFEMRPPLLRRAPSPSLLPASEHKVSPAPRPSSLPILPSGPLYPGLFDIRGSPTGGAGGSADPFAPVFVPPHPGISGGLGGALSGASRSLSPTRLLSLPPDKPFGAKPLGFWTKFDVADWLEWLGLAEHRAQFLDHEIDGSHLPALTKEDYVDLGVTRVGHRMNIDRALKFFLE

SEQ ID NO: 3 - DNA, 20 bp, Artificial Sequence
  primer
cagcctccttcctgcctatc

SEQ ID NO: 4 - DNA, 20 bp, Artificial Sequence
  primer
ggaggatacccagcaccagt

SEQ ID NO: 5 - DNA, 20 bp, Artificial Sequence
  primer
gtccactggtgctgggtatc

SEQ ID NO: 6 - DNA, 20 bp, Artificial Sequence
  primer
gtccactggtgctgggtatc

SEQ ID NO: 7 - DNA, 22 bp, Artificial Sequence
  primer
atctaccgcctagaccaaggtt

SEQ ID NO: 8 - DNA, 22 bp, Artificial Sequence
  primer
tgtggtacagcatcccaagtta

SEQ ID NO: 9 - DNA, 22 bp, Artificial Sequence
  primer
tttcaatggcgtatgtgactcc

SEQ ID NO: 10 - DNA, 21 bp, Artificial Sequence
  primer
cccttggacagcaatgtgttt

SEQ ID NO: 11 - DNA, 20 bp, Artificial Sequence
  primer
tctgcattcacatccattcc

SEQ ID NO: 12 - DNA, 21 bp, Artificial Sequence
  primer
ctgacaagggtgacaataggg

SEQ ID NO: 13 - DNA, 20 bp, Artificial Sequence
  primer
agtcccacattgttcacacg

SEQ ID NO: 14 - DNA, 22 bp, Artificial Sequence
  primer
cttagggtctttctgccttcac

SEQ ID NO: 15 - DNA, 22 bp, Artificial Sequence
  primer
cttggaatgactgaacatttgg

SEQ ID NO: 16 - DNA, 20 bp, Artificial Sequence
  primer
gatggatggatggaggaatg

SEQ ID NO: 17 - DNA, 19 bp, Artificial Sequence
  primer
gctgctgtcctcagtggtg

SEQ ID NO: 18 - DNA, 21 bp, Artificial Sequence
  primer
ccctctgtcttcttccagctc

SEQ ID NO: 19 - DNA, 20 bp, Artificial Sequence
  primer
ttgtcggagtggaaggtttg

SEQ ID NO: 20 - DNA, 20 bp, Artificial Sequence
  primer
ggcatgagggagaaagacag

SEQ ID NO: 21 - DNA, 20 bp, Artificial Sequence
  primer
tctctcccaccatctcttgc

SEQ ID NO: 22 - DNA, 20 bp, Artificial Sequence
  primer
ttggatgagggcctacagag

SEQ ID NO: 23 - DNA, 19 bp, Artificial Sequence
  primer
ctgatgcaccgtcctcttc

SEQ ID NO: 24 - DNA, 20 bp, Artificial Sequence
  primer
atggtcctccaagcctcaag

SEQ ID NO: 25 - DNA, 20 bp, Artificial Sequence
  primer
gctggtaactgtgggaatgc

SEQ ID NO: 26 - DNA, 20 bp, Artificial Sequence
  primer
tttctgcagggtgacaacag

SEQ ID NO: 27 - DNA, 20 bp, Artificial Sequence
  primer
cctaggattcccacgtccac

SEQ ID NO: 28 - DNA, 20 bp, Artificial Sequence
  primer
aagctaattctggcttatcc

SEQ ID NO: 29 - DNA, 20 bp, Artificial Sequence
  primer
ctgtgcagtcatgtgcagtg

SEQ ID NO: 30 - DNA, 20 bp, Artificial Sequence
  primer
aaacctcagctctggtcgtg

SEQ ID NO: 31 - DNA, 19 bp, Artificial Sequence
  primer
ctgaatggatgggtggatg

SEQ ID NO: 32 - DNA, 19 bp, Artificial Sequence
  primer
gggctcagacccaagtcac

SEQ ID NO: 33 - DNA, 19 bp, Artificial Sequence
  primer
gtgaggcctccgtgacttg

SEQ ID NO: 34 - DNA, 18 bp, Artificial Sequence
  primer
aactgggcagccagatcc

SEQ ID NO: 35 - DNA, 20 bp, Artificial Sequence
  primer
ggagggagaggaacatagcc

SEQ ID NO: 36 - DNA, 20 bp, Artificial Sequence
  primer
cacggagaagcagtgctagg

SEQ ID NO: 37 - DNA, 20 bp, Artificial Sequence
  primer
ttccctagcactgcttctcc

SEQ ID NO: 38 - DNA, 20 bp, Artificial Sequence
  primer
cccttcccagagacacacac

SEQ ID NO: 39 - DNA, 19 bp, Artificial Sequence
  primer
gagtggtgagtgggcacag

SEQ ID NO: 40 - DNA, 19 bp, Artificial Sequence
  primer
acaatctcccagcccagtg

SEQ ID NO: 41 - DNA, 20 bp, Artificial Sequence
  primer
gggagattgtgtctccaagc

SEQ ID NO: 42 - DNA, 21 bp, Artificial Sequence
  primer
gaaaccctaggatgtgtgtcg

SEQ ID NO: 43 - DNA, 20 bp, Artificial Sequence
  primer
cttccaccgtcttcacactg

SEQ ID NO: 44 - DNA, 20 bp, Artificial Sequence
  primer
ggattcatggccaagttcac

SEQ ID NO: 45 - DNA, 20 bp, Artificial Sequence
  primer
tgcagtgcacaacctgtacc

SEQ ID NO: 46 - DNA, 20 bp, Artificial Sequence
  primer
ggcagctggaaatagcgtag

SEQ ID NO: 47 - DNA, 19 bp, Artificial Sequence
  primer
ctcccgagatggagacagg

SEQ ID NO: 48 - DNA, 20 bp, Artificial Sequence
  primer
gactccagtcggaggtaggg

SEQ ID NO: 49 - DNA, 19 bp, Artificial Sequence
  primer
ctgttcctgtccaccgacg

SEQ ID NO: 50 - DNA, 20 bp, Artificial Sequence
  primer
gcttttcgaagctgttggag

SEQ ID NO: 51 - DNA, 18 bp, Artificial Sequence
  primer
agggccagcgaagagaac

SEQ ID NO: 52 - DNA, 19 bp, Artificial Sequence
  primer
ccggagcttagagggagtc

SEQ ID NO: 53 - DNA, 19 bp, Artificial Sequence
  primer
agcctatctgccgaaggtg

SEQ ID NO: 54 - DNA, 20 bp, Artificial Sequence
  primer
ccaacctggtttctgtttcc

SEQ ID NO: 55 - DNA, 22 bp, Artificial Sequence
  primer
ccctacccttatgtctctcctc

SEQ ID NO: 56 - DNA, 22 bp, Artificial Sequence
  primer
ccctctgtaatttctcctatcc

SEQ ID NO: 57 - DNA, 22 bp, Artificial Sequence
  primer
gtaacagggagaatcagccaag

SEQ ID NO: 58 - DNA, 22 bp, Artificial Sequence
  primer
aaagatggagaagggagacaca

SEQ ID NO: 59 - DNA, 21 bp, Artificial Sequence
  primer
ttctttcagatttcggctcca

SEQ ID NO: 60 - DNA, 25 bp, Artificial Sequence
  primer
gagacagacagtaaacaagcaagca

SEQ ID NO: 61 - DNA, 22 bp, Artificial Sequence
  primer
tactctgcttggctttctgtcc

SEQ ID NO: 62 - DNA, 23 bp, Artificial Sequence
  primer
ttccacttgccacttctctactg

SEQ ID NO: 63 - DNA, 21 bp, Artificial Sequence
  primer
ttgcactgatggtctgttgag

SEQ ID NO: 64 - DNA, 22 bp, Artificial Sequence
  primer
gggtcaaagcaaacttcatttc

SEQ ID NO: 65 - DNA, 22 bp, Artificial Sequence
  primer
gaaagcatctgagggagagaag

SEQ ID NO: 66 - DNA, 21 bp, Artificial Sequence
  primer
tcttcacatgagggtcaggat

SEQ ID NO: 67 - DNA, 22 bp, Artificial Sequence
  primer
gagtcagccttccatcagaaat

SEQ ID NO: 68 - DNA, 23 bp, Artificial Sequence
  primer
tctgacctctggttggctataag

SEQ ID NO: 69 - DNA, 20 bp, Artificial Sequence
  primer
cgtattcattcacgcaccag

SEQ ID NO: 70 - DNA, 22 bp, Artificial Sequence
  primer
acgtgacaatgatgctgttagg

SEQ ID NO: 71 - DNA, 23 bp, Artificial Sequence
  primer
acccaagcatgaagtgaaatagc

SEQ ID NO: 72 - DNA, 22 bp, Artificial Sequence
  primer
tctttacgtgggtgaattgcat

SEQ ID NO: 73 - DNA, 20 bp, Artificial Sequence
  primer
ttcagcaattcccacccagt

SEQ ID NO: 74 - DNA, 23 bp, Artificial Sequence
  primer
gggtatgcagtgaaagagcagaa

SEQ ID NO: 75 - DNA, 21 bp, Artificial Sequence
  primer
tcaacagaccatcagtgcaag

SEQ ID NO: 76 - DNA, 20 bp, Artificial Sequence
  primer
gcctacctcagtggcaaaga

SEQ ID NO: 77 - DNA, 18 bp, Artificial Sequence
  primer
gactgccgctccaaagtc

SEQ ID NO: 78 - DNA, 20 bp, Artificial Sequence
  primer
gaaggacgctcgtaacttgg

SEQ ID NO: 79 - DNA, 18 bp, Artificial Sequence
  primer
gggaagggcctattctgg

SEQ ID NO: 80 - DNA, 18 bp, Artificial Sequence
  primer
acagtccccatccaatcg

SEQ ID NO: 81 - DNA, 20 bp, Artificial Sequence
  primer
aaccaaccagctcgagaaac

SEQ ID NO: 82 - DNA, 20 bp, Artificial Sequence
  primer
cacgcgatatacggactgtg

SEQ ID NO: 83 - DNA, 20 bp, Artificial Sequence
  primer
agaaagaggctgctcacctg

SEQ ID NO: 84 - DNA, 20 bp, Artificial Sequence
  primer
tctggcctgaatctttgtcc

SEQ ID NO: 85 - DNA, 20 bp, Artificial Sequence
  primer
atgccctacaatccttcgac

SEQ ID NO: 86 - DNA, 22 bp, Artificial Sequence
  primer
aaattgagagcctcatacactg

SEQ ID NO: 87 - DNA, 20 bp, Artificial Sequence
  primer
tcagcttgctcactgtggtc

SEQ ID NO: 88 - DNA, 20 bp, Artificial Sequence
  primer
cctgaacagtcagggcagtc

SEQ ID NO: 89 - DNA, 20 bp, Artificial Sequence
  primer
aagtgcctcctaccttgctg

SEQ ID NO: 90 - DNA, 20 bp, Artificial Sequence
  primer
ttggaattgtgggtcctttc

SEQ ID NO: 91 - DNA, 24 bp, Artificial Sequence
  primer
ttgtgaagagagactaccttggtg

SEQ ID NO: 92 - DNA, 20 bp, Artificial Sequence
  primer
tgggtgcaggtaaggagaag

SEQ ID NO: 93 - DNA, 20 bp, Artificial Sequence
  primer
tgctgatggttaatgcaacg

SEQ ID NO: 94 - DNA, 20 bp, Artificial Sequence
  primer
ctctggaccaactccctgtc